(19) United States
(12) Patent Application Publication
(10) Pub. No.: US 2020/0245846 A1
(43) Pub. Date: Aug. 6, 2020

(54) ENDOSCOPIC SYSTEM, SHEATH AND METHOD OF USING ENDOSCOPIC SYSTEM

(71) Applicant: OLYMPUS CORPORATION, Tokyo (JP)

(72) Inventor: Masahiro Yoshino, Machida-shi (JP)

(73) Assignee: OLYMPUS CORPORATION, Tokyo (JP)

(21) Appl. No.: 16/838,131

(22) Filed: Apr. 2, 2020

Related U.S. Application Data
(63) Continuation of application No. PCT/JP2017/036332, filed on Oct. 5, 2017.

(51) Int. Cl. A61B 1/00; A61B 1/233; A61M 25/10; A61B 1/015; A61B 1/07; A61B 1/01

(52) U.S. Cl. CPC A61B 1/00082; A61B 1/00142; A61B 1/00045; A61B 1/233; A61M 25/10; A61M 2210/0681; A61B 1/015; A61B 1/07; A61B 1/01; A61M 2205/584; A61M 2205/583; A61B 1/00087

(57) ABSTRACT

An endoscopic system includes a sheath portion including an opening, a translucent balloon, an endoscope main body, and a display. The balloon includes a first index group having a plurality of balloon indices. Adjacent two of the plurality of balloon indices are different from each other. The balloon covers the opening and is configured to inflate and deflate. The endoscope main body is configured to be retracted into and projected from the opening. The endoscope main body is located inside the inflated balloon and is configured to observe an interior portion of a sinus. The display is configured to display an image obtained by the endoscope main body.

### CROSS-REFERENCE TO RELATED APPLICATIONS

This application is a Continuation Application of PCT Application No. PCT/JP2017/036332, filed Oct. 5, 2017, the entire contents of which are incorporated herein by reference.

### FIELD

The present invention relates to an endoscopic system for observing the inside of the body of a patient, a sheath and a method for using the endoscopic system.

### BACKGROUND

For example, US2017/0172389A1 discloses an endoscope capable of accessing the paranasal sinuses, etc., and a treatment tool used therefor.

### SUMMARY

According to one aspect of the present disclosure, an endoscopic system includes a sheath portion including an opening, a translucent balloon, an endoscope main body, and a display. The balloon includes a first index group having a plurality of balloon indices. Adjacent two of the plurality of balloon indices are different from each other. The balloon covers the opening and is configured to inflate and deflate. The endoscope main body is configured to be retracted into and projected from the opening. The endoscope main body is located inside the inflated balloon and is configured to observe an interior portion of a sinus. The display is configured to display an image obtained by the endoscope main body.

Advantages will be set forth in the description that follows, and in part will be obvious from the description, or may be learned by practice of the disclosed subject matter. The advantages may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate exemplary embodiments, and together with the general description given above and the detailed description of the embodiments given below, serve to explain the principles of the disclosed subject matter.

FIG. 1 is a schematic diagram illustrating an overall configuration of an endoscopic system according to a first embodiment.

FIG. 2 is a side perspective view showing the vicinity of a distal component of an endoscope insertion section of the endoscopic system shown in FIG. 1.

FIG. 3 is a schematic diagram showing an endoscopic image (image) indicating an internal structure of a balloon of the endoscopic system shown in FIG. 1.

FIG. 4 is a cross-sectional view showing a state in which the insertion apparatus of the endoscopic system shown in FIG. 1 has been inserted into the left nasal cavity and maxillary sinus (paranasal sinus) of the patient.

FIG. 5 is a schematic diagram showing an endoscopic image (image) indicating an internal structure of a balloon of the endoscopic system according to a second embodiment.

FIG. 6 is a cross-sectional view showing a state in which an insertion apparatus of an endoscopic system according to a third embodiment has been inserted into the left nasal cavity and maxillary sinus (paranasal sinus) of the patient.

FIG. 7 is a cross-sectional view showing a state in which an insertion apparatus of an endoscopic system according to a fourth embodiment has been inserted into the left nasal cavity and maxillary sinus (paranasal sinus) of the patient.

FIG. 8 is a cross-sectional view illustrating a balloon, a sheath, and an endoscope main body of an endoscopic system according to a fifth embodiment.

FIG. 9 is a cross-sectional view showing a step of removing the balloon and the endoscope main body from the sheath in the endoscopic system shown in FIG. 8.

### DETAILED DESCRIPTION

### First Embodiment

Hereinafter, a first embodiment of the endoscopic system 11 will be described, with reference to FIGS. 1 to 4.

The endoscopic system 11 includes: an insertion apparatus 12 to be inserted into a nasal cavity 55, a paranasal sinus (maxillary sinus 56), etc. when used; a controller 13 electrically connected to the insertion apparatus 12 via a power line that supplies power thereto and a variety of signal lines; a display 14 connected to the controller 13; a branch path 19 connected to an interior portion of a sheath portion 32, to be described, of the insertion apparatus 12; an air supply and suction device 29 connected to a guide pipe 18 and the sheath portion 32 of the insertion apparatus 12 via the branch path 19; and a syringe 20 connected to the guide pipe 18 and the sheath portion 32 via the branch path 19.

The branch path 19 is configured by a tube that branches off at some midpoint in the shape of a letter “T”. A three-way valve 67 is provided at the portion where the branch path 19 branches off. The three-way valve 67 is capable of switching between a state in which an interior portion of the sheath portion 32 is connected to the air supply and suction device 29, a state in which the interior portion of the sheath portion 32 is connected to the syringe 20, and a state in which the portion where the branch path 19 branches off is occluded.

The branch path 19 is connected in both liquid-tight and air-tight fashion to the interior portion of the sheath portion 32 via a connector, etc. The air supply and suction device 29 includes a vacuum pump 30 and a liquid withdrawal container 31 provided upstream of the vacuum pump 30. The air supply and suction device 29 is capable of generating a negative pressure that suctions the interior portion of the sheath portion 32 by, for example, normally rotating the vacuum pump 30, and providing air to the interior portion of the sheath portion 32 by rotating the vacuum pump 30 in reverse. The air supply and suction device 29 is an example of a suction device.

The syringe 20 is attachable and detachable to and from the branch path 19. The inside of the syringe 20 can be filled with liquids such as various drugs. The user (doctor) can also inject a liquid such as a drug suitable for the condition of the patient into the patient's sinus (paranasal sinus) via the syringe 20, the branch path 19, and the sheath portion 32. The syringe 20 is an example of an injection device. Instead of the syringe 20, a bag, etc. containing therein a liquid such as a drug or a physiological saline, for example, may be connected to the branch path 19.

The insertion apparatus 12 is separated from the display 14 and the controller 13. The display 14 is configured by a general liquid crystal monitor, and is capable of displaying an image acquired by the endoscope 15 as an image 16 (endoscopic image 16).

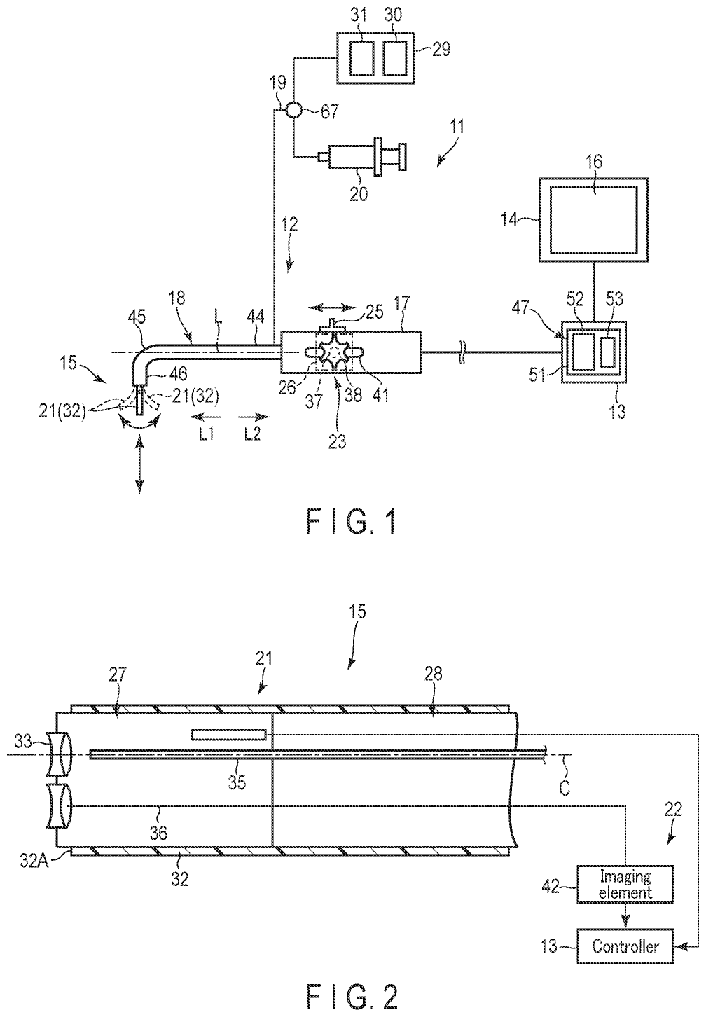

As shown in FIGS. 1 and 2, the insertion apparatus 12 includes: a grip 17 forming an outer shell; a tubular guide pipe 18 (guide) projecting from the grip 17; an endoscope main body 21 allowed to pass through the inside of the guide pipe 18 and the grip 17; an endoscopic imager 22 (imager) provided inside the grip 17; a bending drive 23 provided in the grip 17; a tubular sheath portion 32 covering the periphery of the endoscope main body 21; a pair of wires (pull wires) provided at the right and left sides and straddling the distal end of the sheath portion 32 and the grip 17, and a balloon 61 (see FIG. 4) attached to the distal end of the sheath portion 32 and covering the opening 32A of the sheath portion 32. The grip 17 is formed in, for example, a cylindrical shape, and configures a portion (housing) that is gripped by the user's hand.

In the present embodiment, the endoscope 15 includes an endoscope main body 21 and an endoscopic imager 22, as shown in FIG. 2; however, it may be configured by an endoscope 15 in which these components are integrally formed. In the explanation given below, let us assume that, as shown in FIG. 1, the axial directions of a shaft (to be described later) of the guide pipe 18 are “L”, one of the axial directions L going from the grip 17 (to be described later) toward the elbow 45 is “L1”, and the direction going from the elbow 45 to the grip 17 is “L2”.

The endoscope 15 is configured by a general endoscope (flexible scope) with a known structure; however, it may be configured by a scanning endoscope. The endoscope 15 (endoscope main body 21) is configured to have flexibility. Thus, the endoscope main body 21 can be bent to follow the shape of the guide pipe 18 by being allowed to pass through the guide pipe 18. That is, the endoscope main body 21 is guided by the guide pipe 18, with its direction adjustable. As shown in FIG. 2, a central axis C of the endoscope main body 21 is defined along its longitudinal direction. As shown in FIG. 1, the endoscope main body 21 can be moved along the central axis C, so as to be projected from a projection 46 of the guide pipe 18, and retracted back into the projection 46. Through the action of the advance and retreat mechanism 25 (to be described later), it is also possible to make only the endoscope main body 21 project from the sheath portion 32, or make the endoscope main body 21 retracted back into the sheath portion 32. The endoscope main body 21 is capable of acquiring an image of the optical axis C and the periphery thereof.

As shown in FIG. 2, the endoscope main body 21 includes: a distal component 27 (light receiver) distally located in the central axis C; a flexible tube 28 provided proximal to the distal component 27 in the central axis C; an illumination window 33; a plurality of illumination fibers 35; and a plurality of light-receiving fibers 36. In the present embodiment, the sheath portion 32 has a structure capable of bending in the left-right direction (or up-down direction) together with the endoscope main body 21 (flexible tube 28) retained inside, as shown in FIG. 1. The sheath portion 32 is formed of a flexible, rubber-like elastic body using a water-tight and air-tight material. The sheath portion 32 can be formed of, for example, a resin material such as synthetic rubber.

The grip 17 may be further provided with an advance and retreat mechanism 25 that advances and retreats the endoscope main body 21 in an axial direction L with respect to the guide pipe 18, as shown in FIG. 1. The advance and retreat mechanism 25 is configured by, for example, a knob capable of advancing and retreating the support unit 26. The endoscope main body 21 is guided by the guide pipe 18 when advanced or retreated by the advance and retreat mechanism 25. The user inserts the guide pipe 18 into the patient's sinus (nasal cavity 55, paranasal sinus, etc.) during diagnosis. In this state, through utilization of the advance and retreat mechanism 25, etc., the endoscope main body 21 (distal component 27) can be projected from the projection 46 of the guide pipe 18, the endoscope main body 21 (distal component 27) can be retracted back into the projection 46, or the bending angle of the endoscope main body 21 can be changed using the bending drive 23. Thereby, a desired image 16 in the sinus can be obtained.

The bending drive 23 includes: a support unit 26 housed inside the grip 17 so as to be slidable in the axial direction L of the guide pipe 18; a shaft 37 rotatably supported by the support unit 26; a dial 38 (knob; rotating knob) fixed to one end portion of the shaft 37; and a sprocket (not illustrated) fixed to the other end portion of the shaft 37 inside the casing of the support unit 26. The shaft 37 projects from an elongated hole 41 formed in the grip 17 toward the outside of the grip 17. A chain engageable with the sprocket is connected to an end portion of the wire. As the dial 38 rotates, one of the pair of wires is pulled and the other wire becomes slack, causing the distal end of the sheath portion 32 to be pulled and the sheath portion 32 to be bent toward either the right side or the left side as viewed in FIG. 1. The bend of the sheath portion 32 causes the endoscope main body 21 (flexible tube 28) provided inside to be bent. The bending direction of the sheath portion 32 is an example, and the sheath portion 32 may be bent toward the back side and the front side as viewed in FIG. 1, or bendable in four directions, i.e., upper, lower, right, and left directions, by making the number of the wires four. The wires (linear members) are capable of adjusting the bending angle of the endoscope 15 (endoscope main body 21) when pulled.

As shown in FIG. 2, the illumination fibers 35 are optically connected to a light source provided adjacent to the controller 13. The illumination fibers 35 are capable of emitting illumination light to the exterior portion via a lens, etc. The light-receiving fibers 36 are optically connected to an imaging element 42. Distal ends of the light-receiving fibers 36 are exposed to the exterior portion via a lens, etc. in the vicinity of the distal component 27. Thus, the endoscope 15 is capable of acquiring an image via the light-receiving fibers 36 at the distal component 27. The endoscope 15 is capable of acquiring an image around the optical axis C shown in FIG. 2 via the light-receiving fibers 36.

As shown in FIG. 2, the endoscopic imager 22 includes an imaging element 42 configured by a CCD, a CMOS, etc. The endoscopic imager 22 is capable of acquiring an image obtained by the distal component 27 of the endoscope main body 21. More specifically, the imaging element 42 converts the light from the light-receiving fibers 36 into an electric signal, and sends it to the controller 13.

The light-receiving fibers 36 receive return light from a subject and guide the light to the imaging element 42. The imaging element 42 sends the light received by the light-receiving fibers 36 to the controller 13 as an electric signal. The controller 13 converts the electric signal into an image, processes the image appropriately, and displays it on the display 14.

The controller 13 shown in FIG. 1 includes, for example, a controller main body 47 configured by a general computer, and a power supply 48 provided separately from the controller main body 47. The controller main body 47 is configured by a housing 51, a circuit board 52 built in the housing 51, and a hard disk drive (HDD) 53. A CPU, a ROM, and a RAM are mounted on the circuit board 52.

The controller main body 47 is capable of performing, for example, the following control on each component of the insertion apparatus 12. The controller main body 47 is capable of adjusting an amount of light to be supplied to the illumination fibers 35 through control of the light source. The controller main body 47 is capable of processing an electric signal corresponding to the image acquired by the imaging element 42 of the insertion apparatus 12 into an image, and displaying the image 16 (endoscopic image) on the display 14.

As shown in FIG. 1, the guide pipe 18 (guide) as a whole is formed substantially in the shape of a letter “L”, and is formed in a tubular shape (cylindrical shape) bent in an elbow shape at some midpoint. The guide pipe 18 includes a shaft 44 attached to the grip 17 at one end, an elbow 45 provided at the other end of the shaft 44, and a projection 46 projecting from the elbow 45 in a direction intersecting a direction in which the shaft 44 extends (i.e., a lateral direction).

The endoscope main body 21 can be allowed to pass through the inside of the guide pipe 18. An inner wall of the guide pipe 18 is capable of guiding the endoscope main body 21, which advances and retreats along the central axis C. It is preferable that the guide pipe 18 be provided so as to be fixed to, for example, the grip 17; however, it may be rotatable around the axial direction L with respect to the grip 17. In this case, the grip 17 may be provided with a rotation knob for rotating the guide pipe 18 around the axial direction L.

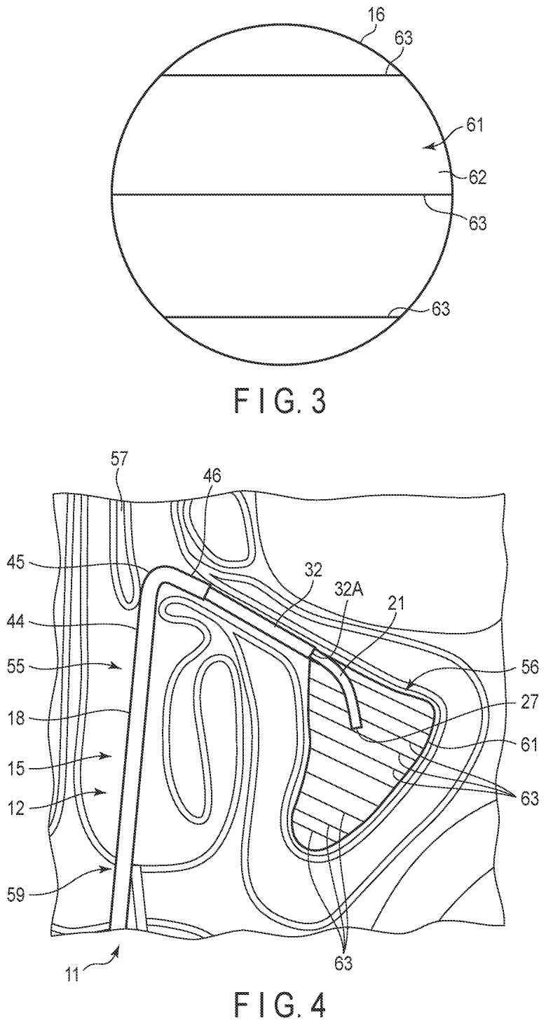

As shown in FIGS. 3 and 4, the balloon 61 is formed of a rubber-like elastic material, such as synthetic rubber, etc., so as to be both inflatable and deflatable. The balloon 61 has translucency, and allows for observation of the inside of the maxillary sinus 56 via the endoscope main body 21 from within the balloon 61, as will be described later. In the present embodiment, the balloon 61 is detachably attached to, for example, a distal end of the sheath portion 32. Moreover, the balloon 61 may be attached to the distal component 27 of the endoscope main body 21, or the neighborhood thereof.

The balloon 61 includes a balloon main body 62, and a plurality of first indices 63 (a first index group) formed on an inner peripheral surface of a balloon main body 62. The first indices 63 are formed by printing or applying paint on an inner peripheral surface of the balloon main body 62. Since the balloon 61 is translucent, the first indices 63 may be formed on an outer peripheral surface of the balloon main body 62.

As shown in FIG. 3, the first indices 63 are a plurality of straight lines arranged at predetermined distances or predetermined intervals. It is preferably that the plurality of first indices (balloon indices) 63 are adjacent to one another side by side in a direction intersecting a central axis LA of the projection 46 of the guide pipe 18, for example, in the direction orthogonal to the central axis LA. Thus, the first indices 63 are arranged in a so-called contour line in the balloon 61. Even though the first indices 63 are arranged at, for example, equal intervals, the interval between the first indices 63 may be changed. The first indices 63 are configured, for example, in parallel with each other. In the first embodiment, the colors of the straight lines constituting the first indices 63 are, for example, different from each other. Adjacent first indices 63 of the plurality of first indices 63 are different from each other. Non-adjacent first indices 63 may have different appearances or the same appearance. The straight lines constituting the first indices 63 can be in colors selected from various colors; as an example, they may be rendered distinguishable from one another using colors such as red, yellow, green, blue, etc. in the order of proximity to the distal end of the sheath portion 32. The user can distinguish the adjacent first indices 63 by observing the images 16 displayed on the display 14. As a matter of course, the straight lines may be colored with colors other than these to render the straight lines distinguishable from one another.

The configuration for rendering the straight lines of the first indices 63 distinguishable from one another can, instead of color coding, also be provided by, for example, making the densities of shading of the lines different from one another. In this case, gradations may be formed in such a manner that, for example, the line is in a light color at a position close to the distal end of the sheath portion 32, and the color of the line gradually darkens as distance from the distal end of the sheath portion 32 increases. The gradations of the colors of the lines may be formed conversely, in such a manner that the line is in a dark color at a position close to the distal end of the sheath portion 32, and the color of the line gradually lightens as distance from the distal end of the sheath portion 32 increases.

Moreover, the straight lines constituting the first indices 63 may be rendered distinguishable from one another by varying the thickness. In this case, for example, the lines may be formed in such a manner that the line has a small thickness at a position close to the distal end of the sheath portion 32, and the thickness of the line gradually increases as distance from the distal end of the sheath portion 32 increases. The thicknesses of the lines may be configured conversely, in such a manner that the line has a large thickness at a position close to the distal end of the sheath portion 32, and the thickness of the line gradually decreases as distance from the distal end of the sheath portion 32 increases.

The straight lines constituting the first indices 63 may be rendered distinguishable from one another by making them of different line types. In this case, the lines may be formed in such a manner that, for example, a sparsely-dashed straight line is formed using intermittently-formed dashes at a position close to a distal end of the sheath portion 32, and the line gradually becomes more densely-dashed and ultimately continuous, in the order of a three-dot chain line, a two-dot chain line, a one-dot chain line, and a continuous line, as distance from the distal end of the sheath portion 32 increases. The variation in the line type may be formed conversely, in such a manner that a densely-dashed or continuous straight line is formed at a position close to the distal end of the sheath portion 32, and the line becomes gradually more sparsely-dashed, in the order of a one-dot chain line, a two-dot chain line, a three-dot chain line, and a sparsely-dashed line, as distance from the distal end of the sheath portion 32 increases.

Furthermore, the first indices 63 can be provided not only by a plurality of straight lines, but also by combining character information with straight lines. In this case, as the first indices 63, the characters “parietal side” may be attached to the neighborhood of a straight line at a position close to the distal end of the sheath portion 32, and the characters “jaw side” may be attached to the neighborhood of a straight line at a position farthest from the distal end of the sheath portion 32. In this case, “parietal side” corresponds to the parietal side of the patient, and “jaw side” corresponds to the jaw side of the patient.

Furthermore, the character information of the first indices 63 may be displayed not only on the balloon 61 but also in the image 16 of the display 14. In this case, the controller 13 identifies an approximate position in the maxillary sinus 56 by performing image recognition using the first indices 63 as markers. Thereby, characters such as “parietal side”, “jaw side”, “anterior side”, “posterior side”, etc. can be displayed in the image 16 in the neighborhood of straight lines constituting the first indices 63. Furthermore, image recognition may be performed using the first indices 63 as markers, and an approximate position of the distal component 27 may be displayed on a CT image obtained from a CT image acquisition device provided separately. It should be noted that the examples of the configuration of the first indices 63, the display in the image 16 additionally performed on the first indices 63, and the display of the position in the CT image described above are not mutually exclusive and the endoscopic system 11 may be provided by appropriately combining them, as a matter of course.

Subsequently, an observation method using the endoscopic system 11 of the present embodiment will be described, with reference to FIGS. 3 and 4.

The maxillary sinus 56, which is one of the paranasal sinuses of the human body, constitutes a branch portion extending laterally from the nasal cavity 55, with respect to the nasal cavity 55 located at a substantially central portion of the face and extending in an anteroposterior direction. The nasal cavity 55 and the maxillary sinus 56 are separated by an organ known as the middle nasal concha 57, etc. In order to observe the maxillary sinus 56 with the endoscopic system 11, the endoscope main body 21 is inserted into the middle of the nasal cavity 55 as viewed in the anteroposterior direction, and the projection 46 of the guide pipe 18 is directed lateral to the patient. In this state, the sheath portion 32 and the endoscope main body 21 are made to project from the projection 46, thus allowing the sheath portion 32 and the endoscope main body 21 to be located in the vicinity of the entrance of the maxillary sinus 56. Through the sending of a gas such as air to the sheath portion 32 via the air supply and suction device 29, the balloon 61 located at the distal end of the sheath portion 32 can be expanded. The fluid (working fluid) for expanding the balloon 61 is not limited thereto, and the balloon 61 may be inflated with a liquid injected via the syringe 20, as a matter of course. This allows the maxillary sinus 56 of the patient to be expanded, thus making it easily observable for the user (doctor). In this state, the user further operates the advance and retreat mechanism 25 to make the endoscope main body 21 project, thus allowing the distal component 27 to be located in the maxillary sinus 56 inside the balloon 61. In this state, the user can observe an interior state of the maxillary sinus 56. At this time, no landmarks are generally present in the interior portion of the maxillary sinus 56; however, landmarks can be artificially formed in the maxillary sinus 56 using the first indices 63 of the balloon 61. Moreover, since the balloon has translucency, the balloon does not obstruct the visual field of the endoscope main body 21. This allows the user to observe the interior portion of the maxillary sinus 56, while referring to the first indices 63 serving as landmarks. Thereby, the user can reliably observe a site to be observed in the maxillary sinus 56, without the problem of losing a grasp of the direction of the image 16 displayed on the display 14. It is thereby possible for the user to observe the portion which is inflamed, and the portion filled with pus.

According to the first embodiment, the following can be said. An endoscopic system 11 includes: a sheath portion 32 including an opening 32A; a translucent balloon 61 including a plurality of first indices 63 different from each other, and covers the opening 32A and configured to inflate and deflate; an endoscope main body 21 configured to be retracted into and projected from the opening 32A so as to be located inside the inflated balloon 61, and configured to observe an interior portion of a sinus; and a display 14 which is configured to display an image 16 obtained by the endoscope main body 21. According to the first embodiment, a sheath which allows an endoscope 15 to be inserted therethrough, includes: a sheath portion 32 including an opening 32A; and a balloon 61 having translucency covering the opening 32A and configured to inflate and deflate, the balloon 61 including a plurality of first indices 63 which is different from each other.

With this configuration, in which the interior portion of the sinus can be expanded by the balloon 61, it is possible, during observation of the interior portion of the sinus using the endoscope main body 21, to make the visibility of the interior portion of the sinus preferable, since adhesion of, in particular, tissues that are adhered to each other can be eliminated. At this time, since the first indices 63 are provided in the balloon 61, the interior portion of the sinus can be observed with reference to the first indices 63. This prevents occurrence of the problem of losing a grasp of the direction in the actual space to which the direction in the image 16 corresponds, when the user is observing the interior portion of the sinus while looking at the image 16 displayed on the display 14. It is thereby possible to provide a user-friendly endoscopic system 11.

The first indices 63 are a plurality of straight lines arranged at predetermined intervals. Moreover, the first indices 63 are parallel to each other. Furthermore, the first indices 63 are different from each other in at least one of the color, the density of shading of the line, the thickness, and the line type. With the above-described configuration, it is possible to provide the first indices 63 with ease and at low cost.

According to the present embodiment, the method using the endoscopic system 11 is a method using the endoscopic system 11 comprising: a sheath portion 32 including an opening 32A; a balloon 61 including a plurality of first indices 63; and an endoscope main body 21 housed inside the opening 32A, in which the balloon 61 is inflated to make the endoscope main body 21 projected from the opening 32A and allow the endoscope main body 21 to be located inside the inflated balloon 61, allowing for observation of the interior portion of the sinus with the endoscope main body 21 with reference to the first indices 63.

With this configuration, in which the interior portion of the sinus can be observed with reference to the first indices 63, it is possible to prevent the user viewing an image obtained by the endoscope main body 21 from experiencing the problem of losing a grasp of the direction in the image. It is thereby possible to provide a user-friendly endoscopic system 11.

### Second Embodiment

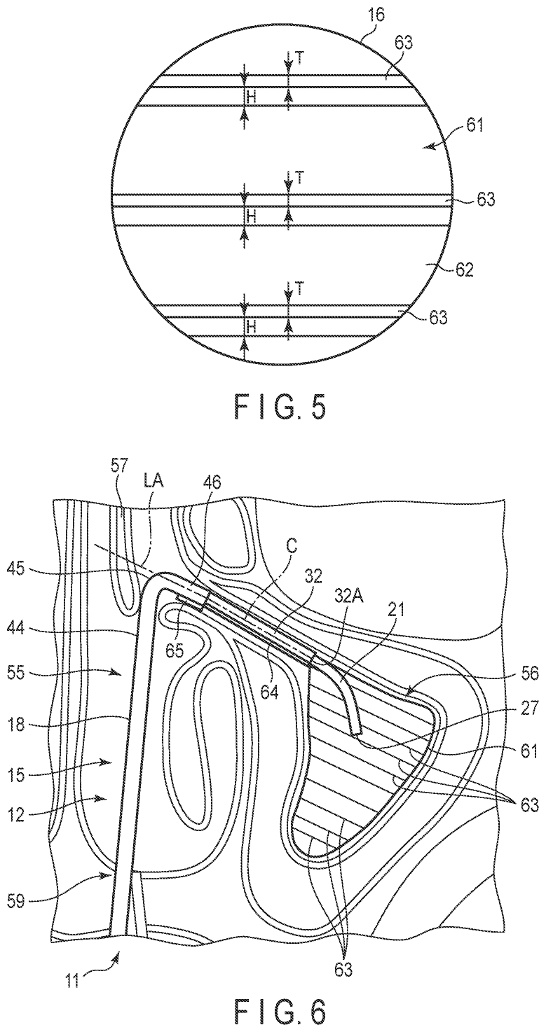

An endoscopic system 11 according to a second embodiment will be described with reference to FIG. 5. In the second embodiment, the configuration of a plurality of first indices 63 is different from that of the first embodiment; however, the other portions are common to the first embodiment. In the following, portions different from those of the first embodiment will be mainly described, and descriptions and/or illustrations of portions common to the first embodiment will be omitted.

Each of the first indices 63 is configured by a convex portion projecting linearly from the balloon main body 62 toward the inside of the balloon 61. The first indices 63 (convex portions) are arranged at, for example, predetermined intervals. Even though the first indices 63 are arranged at, for example, equal intervals, the interval between the first indices 63 may be changed. As shown in FIG. 5, the first indices 63 are configured, for example, in parallel with each other. The first indices 63 are configured, for example, to have the same thickness.

In the first embodiment, the colors of the straight lines constituting the first indices 63 (convex portions) are different from each other. The straight lines (convex portions) constituting the first indices 63 can be in colors selected from various colors; as an example, they may be rendered distinguishable from one another using colors such as red, yellow, green, blue, etc. in the order of proximity to the distal end of the sheath portion 32. As a matter of course, the first indices 63 may be colored with colors other than these to make the first indices 63 distinguishable from one another.

The configuration of making the first indices 63 (convex portions) distinguishable from one another can also be achieved by, for example, making the densities of shading of the colors added to the first indices 63 different from one another, instead of the color coding. In this case, gradations may be formed in such a manner that, for example, the first index 63 is in a light color at a position close to the distal end of the sheath portion 32, and the color of the first index 63 gradually darkens as distance from the distal end of the sheath portion 32 increases. The gradations of the colors of the first indices 63 may be formed conversely, in such a manner that the first index 63 is in a dark color at a position close to the distal end of the sheath portion 32, and the colors of the first indices 63 gradually lighten as distance from the distal end of the sheath portion 32 increases.

Moreover, the first indices 63 (convex portions) may be rendered distinguishable from one another by varying a thickness T or a height H. In this case, the first indices 63 may be formed in such a manner that, for example, a first index 63 (convex portion) at a position close to the distal end of the sheath portion 32 has a small thickness T or height H, and the thickness T or height H of the first indices 63 gradually increases as distance from the distal end of the sheath portion 32 increases. Conversely, a first index 63 at a position close to the distal end of the sheath portion 32 may have a large thickness T or height H, and the thickness T or height H of the first index 63 may be gradually decreased as distance from the distal end of the sheath portion 32 increases.

The first indices 63 (convex portions) may be rendered distinguishable from one another by making them of different line types. In this case, the convex portions may be formed in such a manner that, for example, a sparsely-convexed portion (sparsely-dashed convex portion) is provided using intermittently-formed convexes at a position close to a distal end of the sheath portion 32, and the convex portion becomes gradually more densely-convexed, in the order of a three-dot chain line, a two-dot chain line, a one-dot chain line, and a continuous line as distance from the distal end of the sheath portion 32 increases. The convex portions may be formed conversely, in such a manner that, for example, a densely-convexed straight line (continuous straight line) is provided at a position close to a distal end of the sheath portion 32, and the convex portion becomes gradually more sparsely-convexed, in the order of a one-dot chain line, a two-dot chain line, a three-dot chain line, and a sparsely-convexed line as distance from the distal end of the sheath portion 32 increases.

Furthermore, the first indices 63 can be provided by combining character information with a plurality of straight lines, instead of the linear convex portions. In this case, the characters “parietal side” may be attached to the neighborhood of a straight line at a position close to the distal end of the sheath portion 32, and the characters “jaw side” may be attached to the neighborhood of a straight line at a position farthest from the distal end of the sheath portion 32. Moreover, characters such as “parietal side”, “jaw side”, “anterior side”, and “posterior side” may be displayed in the image 16 in the neighborhood of the first indices 63 in the image 16, subsequent to image recognition using the first indices 63 as markers to identify an approximate position in the maxillary sinus 56. Furthermore, image recognition may be performed using the first indices 63 as markers, and an approximate position of the distal component 27 may be displayed on a CT image obtained from a CT image acquisition device provided separately. It should be noted that the examples of the configuration of the first indices 63, the display in the image 16 additionally performed on the first indices 63, and the display of the position in the CT image described above are not mutually exclusive, and the endoscopic system 11 may be provided by appropriately combining them, as a matter of course.

The present embodiment is capable of exhibiting substantially the same operation as that of the first embodiment.

According to the present embodiment, a plurality of first indices 63 are a plurality of convex portions projecting linearly toward the interior portion of the balloon 61 and arranged at predetermined intervals. The first indices 63 are parallel to each other. The first indices 63 are different from each other in at least one of the color, the density of shading of the color, the thickness, and the type of the convex portion. With the above-described configuration, it is possible to provide the first indices 63 with high visibility and with a simple structure.

### Third Embodiment

An endoscopic system 11 according to a third embodiment will be described, with reference to FIG. 6. In the third embodiment, the structures of a guide pipe 18 and a sheath portion 32 are different from those of the first embodiment; however, the other portions are common to the first embodiment. In the following, portions different from those of the first embodiment will be mainly described, and descriptions and/or illustrations of portions common to the first embodiment will be omitted.

The guide pipe 18 includes a third index 65 formed in a linear shape on an outer peripheral surface of a projection 46. In the present embodiment, the third index 65 is provided, for example, at a position opposed to a shaft 44 of the guide pipe 18. The third index 65 is provided in parallel with a central axis LA of the projection 46 of the guide pipe 18. The third index 65 is formed by, for example, printing or applying a paint on an outer peripheral surface of the projection 46.

The sheath portion 32 includes a second index 64 formed linearly on a portion of its outer peripheral surface. The second index 64 is provided in parallel with a central axis of the sheath portion 32 (a central axis C of an endoscope main body 21). The second index 64 is formed by, for example, printing or applying a paint on an outer peripheral surface of the sheath portion 32. In the present embodiment, the endoscope main body 21 is provided so as to be fixed to the sheath portion 32, and does not rotate around the central axis C with respect to the sheath portion 32.

Subsequently, an observation method using the endoscopic system 11 of the present embodiment will be described with reference to FIG. 6.

In the present embodiment, the second index 64 of the sheath portion 32 is positioned with respect to the third index 65 of the guide pipe 18, prior to insertion of the insertion apparatus 12 into the nasal cavity 55. Thereby, the position of the sheath portion 32 is correctly set with respect to the guide pipe 18, and the image (image 16) obtained from the endoscope main body 21 can be correctly displayed on the display 14 at a scheduled angle.

After the endoscope main body 21 is inserted into the middle of the nasal cavity 55 as viewed in the anteroposterior direction, the projection 46 of the guide pipe 18 is directed lateral to the patient. In this state, the sheath portion 32 and the endoscope main body 21 are made to project from the projection 46, thus allowing the sheath portion 32 and the endoscope main body 21 to be located in the vicinity of the entrance of the maxillary sinus 56. Through the sending of air to the sheath portion 32 via the air supply and suction device 29, the balloon 61 located at the distal end of the sheath portion 32 can be expanded. This allows the maxillary sinus 56 of the patient to be expanded, thus making it easily observable for the user (doctor). In this state, the user further operates the advance and retreat mechanism 25 to make the endoscope main body 21 project, thus allowing the distal component 27 to be located in the maxillary sinus 56 inside the balloon 61. Landmarks can be artificially formed in the maxillary sinus 56 using the first indices 63 of the balloon 61. At this time, since the angle of the sheath portion 32 with respect to the guide pipe 18 is determined, the position of the balloon 61 attached to the distal end of the sheath portion 32 with respect to the guide pipe 18 can be correctly set. Thereby, the first indices 63 of the balloon 61 can be disposed at a correct position with respect to the maxillary sinus 56 of the patient. Thus, it is possible for the user to more reliably observe a site to be observed in the maxillary sinus 56, while referring to the first indices 63 placed with high accuracy.

According to the present embodiment, the endoscopic system 11 further includes a guide that guides the insertion direction of the sheath portion 32, the balloon 61 is attached to the sheath portion 32, the sheath portion 32 includes a second index 64, and the guide includes a third index 65 for positioning with respect to the second index 64. With this configuration, since the sheath portion 32 can be positioned with respect to the guide using the second index 64 and the third index 65, the balloon 61 can be disposed in a sinus at a scheduled angle. Thereby, the first indices 63 can be more accurately located in the sinus, allowing the user who refers thereto to more accurately identify the position in the sinus. It is thereby possible to provide a more user-friendly endoscopic system 11.

### Fourth Embodiment

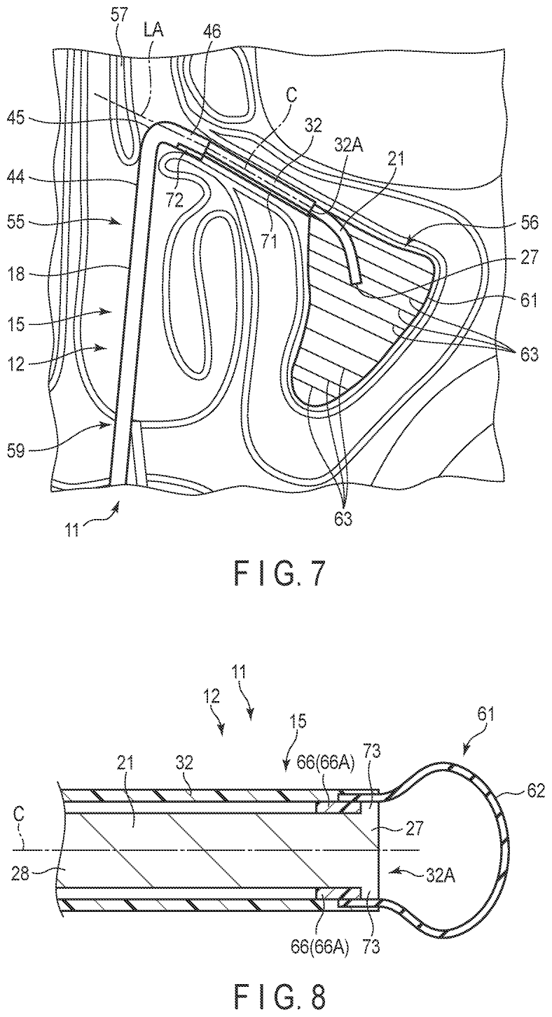

An endoscopic system 11 according to a fourth embodiment will be described, with reference to FIG. 7. In the fourth embodiment, the structures of the guide pipe 18 and the sheath portion 32 are different from those of the first embodiment; however, the other portions are common to the first embodiment. In the following, portions different from those of the first embodiment will be mainly described, and descriptions and/or illustrations of portions common to the first embodiment will be omitted.

The guide pipe 18 includes, on an inner peripheral surface of the projection 46, a second engagement 72 formed in a concave shape depressed from the peripheral portion in such a manner that the concave portion extends linearly. In the present embodiment, the second engagement 72 is provided, for example, at a position facing the shaft 44 of the guide pipe 18. The second engagement 72 is provided in parallel with the central axis LA of the projection 46 of the guide pipe 18. The second engagement 72 may be formed in a slot shape or a groove shape so as to penetrate part of the guide pipe 18.

The sheath portion 32 includes a first engagement 71 on a portion of its outer peripheral surface. The first engagement 71 is formed as a convex portion extending linearly (in a rail shape). The first engagement 71 is formed, for example, so as to project outward from the outer peripheral surface of the sheath portion 32. The first engagement 71 is provided in parallel with the central axis of the sheath portion 32 (the central axis C of the endoscope main body 21). The first engagement 71 fits into the inside of the second engagement 72 so as to be slidable. Thus, the second engagement 72 is capable of preventing the first engagement 71 and the sheath portion 32 from rotating around the central axis C. It is to be noted that the first engagement 71 and the second engagement 72 may take any shapes; as a matter of course, the first engagement 71 may be formed as a concave portion and the second engagement 72 may be formed as a convex portion.

In the present embodiment, the endoscope main body 21 is provided so as to be fixed to the sheath portion 32, and does not rotate around the central axis C with respect to the sheath portion 32.

Subsequently, an observation method using the endoscopic system 11 of the present embodiment will be described, with reference to FIG. 7.

In the present embodiment, prior to inserting the insertion apparatus 12 into the nasal cavity 55, the second engagement 72 of the guide pipe 18 is fitted into the first engagement 71 of the sheath portion 32. Thus, the position of the sheath portion 32 is set correctly with respect to the guide pipe 18, and the image (image 16) obtained from the endoscope main body 21 is correctly displayed on the display 14 at a scheduled angle.

After the endoscope main body 21 is inserted into the middle of the nasal cavity 55 as viewed in the anteroposterior direction, the projection 46 of the guide pipe 18 is directed lateral to the patient. In this state, the sheath portion 32 and the endoscope main body 21 are made to project from the projection 46, thus allowing the sheath portion 32 and the endoscope main body 21 to be located in the vicinity of the entrance of the maxillary sinus 56. Through the sending of air to the sheath portion 32 via the air supply and suction device 29, the balloon 61 located at the distal end of the sheath portion 32 can be expanded. This allows the maxillary sinus 56 of the patient to be expanded, thus making it easily observable for the user (doctor). In this state, the user further operates the advance and retreat mechanism 25 to make the endoscope main body 21 project, thus allowing the distal component 27 to be located in the maxillary sinus 56 inside the balloon 61. Landmarks can be artificially formed in the maxillary sinus 56 using the first indices 63 of the balloon 61. At this time, since the angle of the sheath portion 32 with respect to the guide pipe 18 is determined by the second engagement 72 and the first engagement 71, the position of the balloon 61 attached to the distal end of the sheath portion 32 with respect to the guide pipe 18 can be correctly set. Thereby, the first indices 63 of the balloon 61 is disposed at a correct position with respect to the maxillary sinus 56 of the patient. Thus, it is possible for the user to more reliably observe a site for observation in the maxillary sinus 56, while referring to the first indices 63 serving as landmarks.

According to the present embodiment, a guide for guiding the insertion direction of the sheath portion 32 is further provided, the balloon 61 is attached to the sheath portion 32, the sheath portion 32 includes a first engagement 71, the guide includes a second engagement 72 that engages with the first engagement 71, and the second engagement 72 permits movement of the sheath portion 32 in a longitudinal direction of the sheath portion 32 and restricts axial rotation of the sheath portion 32.

With this configuration, it is possible to prevent the sheath portion 32 from axially rotating with respect to the guide. This allows the balloon 61 to be disposed in a sinus at a scheduled angle. Thereby, the first indices 63 can be located more accurately in the sinus, and the user who refers thereto can more accurately identify its position in the sinus. It is thereby possible to provide a more user-friendly endoscopic system 11.

### Fifth Embodiment

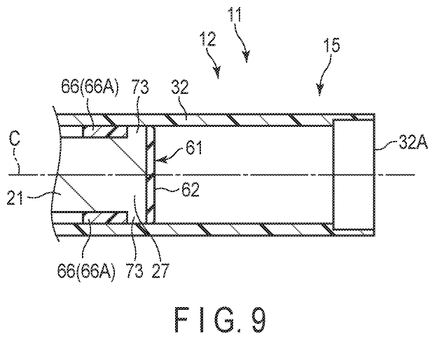

An endoscopic system 11 according to a fifth embodiment will be described with reference to FIGS. 8 and 9. In the fifth embodiment, the attachment structure of the balloon 61 is different from that of the first embodiment; however, the other portions are common to the first embodiment. In the following, portions different from those of the first embodiment will be mainly described, and descriptions and/or illustrations of portions common to the first embodiment will be omitted.

An endoscope main body 21 includes a distal component 27 and a flange 73 formed in a flange shape in the periphery of the distal component 27.

The balloon 61 includes a balloon main body 62 and an attachment 66 provided at an end portion of the balloon main body 62. The attachment 66 is formed in a ring shape using a resin material, etc. The balloon main body 62 and the attachment 66 are formed integrally. Alternatively, the attachment 66 may be formed in a single ring shape by allowing engagement between a first ring member (inner ring member) and a second ring member (outer ring member). In this case, the first ring member engages with the second ring member with an end of the balloon main body 62 interposed between the first ring member and the second ring member. With such a structure, the balloon main body 62 and the attachment 66 may be formed integrally.

The attachment 66 is engageable with the flange 73. Thus, in the present embodiment, the balloon 61 is attached to the endoscope main body 21. The attachment 66 includes an attachment main body 66A, and a plurality of ventilation holes penetrating therethrough. Each of the ventilation holes penetrates the attachment main body 66A in the central axis C.

When the balloon 61 is inflated, air is sent into the sheath portion 32 by the air supply and suction device 29, in such a manner that air is sent into the interior portion of the balloon main body 62 via the ventilation hole. When the balloon 61 is deflated, the air in the interior portion of the balloon main body 62 is exhausted via the ventilation holes, through the suctioning of the inside of the sheath portion 32 via the air supply and suction device 29.

Subsequently, an observation method using the endoscopic system 11 of the present embodiment will be described, with reference to FIGS. 8 and 9.

After the endoscope main body 21 is inserted into the middle of the nasal cavity 55 as viewed in the anteroposterior direction, the projection 46 of the guide pipe 18 is directed lateral to the patient. In this state, the sheath portion 32 and the endoscope main body 21 are made to project from the projection 46, thus allowing the sheath portion 32 and the endoscope main body 21 to be located in the vicinity of the entrance of the maxillary sinus 56. Through the sending of air to the sheath portion 32 via the air supply and suction device 29, the balloon 61 located at the distal end of the sheath portion 32 can be expanded, as shown in FIG. 8. The balloon 61 can be further inflated in the maxillary sinus 56 from the state shown in FIG. 8. This allows the maxillary sinus 56 of the patient to be expanded, thus making it easily observable for the user (doctor). In this state, the user further operates the advance and retreat mechanism 25 to make the endoscope main body 21 project, thus allowing the distal component 27 to be located in the maxillary sinus 56 inside the balloon 61. At this time, it is preferable that the sheath portion 32 is also disposed in the maxillary sinus 56 (sinus). Landmarks can be artificially formed in the maxillary sinus 56 using the first indices 63 of the balloon 61. Thus, it is possible for the user to reliably observe a site to be observed in the maxillary sinus 56, while referring to the first indices 63 serving as landmarks.

It is possible for the user to remove the balloon 61 and perform the procedures the interior portion of the maxillary sinus 56. For example, the balloon 61 is deflated by the air supply and suction device 29, as shown in FIG. 9. Furthermore, by pulling out the endoscope main body 21 from the sheath portion 32, the endoscope main body 21 can be removed together with the balloon 61. At this time, the sheath portion 32 can retain the position adopted prior to the removal of the balloon 61 and the endoscope main body 21. The user removes the attachment 66 from the flange 73 after the removal of the endoscope main body 21 from the sheath portion 32. Thereby, the balloon 61 is removed from the endoscope main body 21.

Thereafter, the endoscope main body 21 is inserted back into the sheath portion 32, allowing for visual recognition of the interior portion of the maxillary sinus 56. In this state, the user can, for example, suction and remove the pus with which the maxillary sinus 56 is filled. If the endoscope main body 21 interferes with the operation of the suction and removal of the pus, the suction and removal can be performed without disposing the endoscope main body 21 inside the sheath. In this case, since the sheath portion 32 retains the position adopted prior to the removal of the endoscope main body 21, the sheath portion 32 does not deviate from the position where the pus is present. Alternatively, a channel may be provided in the endoscope main body 21 so as to penetrate it in the central axis C (axial direction), in such a manner that the suction and removal of the pus can be performed via the channel. In this case, the channel is connected to the branch path 19 and the air supply and suction device 29.

Alternatively, after the user removes the balloon 61 from the endoscope main body 21, the user inserts the endoscope main body 21 back into the sheath portion 32, allowing for visual recognition of the interior portion of the maxillary sinus 56. In this state, instead of the suction and removal of the pus, a liquid (drug) can be injected into the maxillary sinus 56 via the sheath portion 32, for example. The injection of the liquid (drug) is performed in such a manner that the liquid (drug) is injected into the paranasal sinus (maxillary sinus 56) via the sheath portion 32, through the operation of the syringe 20. If the endoscope main body 21 interferes with the operation of the injection of the liquid (drug), the injection of the liquid (drug) can be performed without disposing the endoscope main body 21 inside the sheath. In this case, since the sheath portion 32 retains the position adopted prior to the removal of the endoscope main body 21, the sheath portion 32 does not deviate from the site into which the liquid (drug) is to be injected. Alternatively, the above-described channel may be provided in the endoscope main body 21, and the liquid (drug) may be injected via the channel. In this case, the channel is connected to the branch path 19 and the syringe 20.

With the foregoing, it is possible, in the present embodiment, not only to observe the interior portion of the paranasal sinus (maxillary sinus 56) using the endoscopic system 11, but also to perform the procedures the maxillary sinus 56 using the endoscopic system 11.

In the present embodiment, the attachment 66 is engaged with the flange 73; however, the structure of fixing the balloon 61 to the distal component 27 is not limited thereto. Magnets may be respectively provided in the attachment 66 and the endoscope main body 21, in such a manner that the balloon 61 is fixed to the distal component 27 by the magnetic force. Moreover, the shape of the attachment 66 is not limited to a ring shape, and may be, for example, a tube shape extending to the grip 17. In this case, the balloon 61 can be removed by pulling out the tube-shaped attachment 66 from the side of the grip 17.

According to the present embodiment, a balloon 61 is attached to an endoscope main body 21, and the endoscope main body 21 is, together with the balloon 61, removable from within the sheath portion 32 in a direction opposite to an opening 32A. With this configuration, it is possible for the user to easily remove the balloon 61 when the balloon 61 interferes.

The endoscopic system 11 includes a suction device that is connectible to a sheath portion 32 from which the endoscope main body 21 and the balloon 61 have been removed, and is capable of suctioning the interior portion of the sinus via the sheath portion 32. The endoscopic system 11 includes an injection device that is connectible to the sheath portion 32 from which the endoscope main body 21 and the balloon 61 have been removed, and is capable of injecting a liquid into the sinus via the sheath portion 32. With this configuration, it is possible not only to observe the interior portion of the sinus using the endoscopic system 11, but also to perform an intra-sinus suctioning procedure and a liquid injection procedure using the endoscopic system 11. Thus, when the procedures are performed after observation, the endoscope main body 21 need not be replaced with instruments designed for various procedures to be performed, and it is possible to provide a user-friendly endoscopic system 11.

In the method of using the endoscopic system 11, the endoscope main body 21 is removed, together with the balloon 61, from within the sheath portion 32 in a direction opposite to the opening 32A, and a predetermined procedures is performed on the interior portion of the sinus via the sheath portion 32 from which the endoscope main body 21 and the balloon 61 have been removed. With this configuration, in which the endoscope main body 21 is removed, together with the balloon 61, from within the sheath portion 32, it is possible to perform a predetermined procedure through the effective utilization of the remaining sheath portion 32. In addition, the position of the sheath portion 32 does not deviate from the position at which observation is being performed with the endoscope main body 21. It is thereby possible to perform a predetermined procedure on the position at which observation is being performed with the endoscope main body 21, thus further enhancing the user's convenience.

The predetermined procedure is to suction the content in the sinus via the sheath portion 32. With this configuration, it is possible to suction the content in the sinus via the sheath portion 32, while maintaining the position at which observation is being performed with the endoscope main body 21. Thus, suction can be performed efficiently without causing a deviation in the position of the sheath portion 32 prior to the suction, and the sheath portion 32 remaining after the removal of the balloon 61 and the endoscope main body 21 can be effectively utilized. Thus, convenience for the user can be further enhanced.

The predetermined procedure is to inject a liquid into the sinus via the sheath portion 32. With this configuration, it is possible to inject a liquid into a sinus via the sheath portion 32, while maintaining the position at which observation is being performed with the endoscope main body 21. Thus, liquid injection can be performed efficiently without causing a deviation in the position of the sheath portion 32 prior to the suction, and the sheath portion 32 remaining after the removal of the balloon 61 and the endoscope main body 21 can be effectively utilized. Thus, convenience for the user can be further enhanced.

Note that the balloon 61 may be inflated and deflated by water, etc. instead of air, and the type of the fluid for inflating and deflating the balloon 61 is not limited to a particular kind. As a matter of course, a single endoscopic system 11 may be provided by appropriately combining the constituent elements described in the above-described embodiments.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscopic system comprising:
a sheath portion including an opening;
a translucent balloon including a first index group having a plurality of balloon indices, adjacent two of the plurality of balloon indices being different from each other, and the balloon covering the opening and configured to inflate and deflate;
an endoscope main body configured to be retracted into and projected from the opening, the endoscope main body being located inside the inflated balloon and configured to observe an interior portion of a sinus; and
a display configured to display an image obtained by the endoscope main body.

2. The endoscopic system according to claim 1, wherein:
each of at least two of the plurality of balloon indices of the first index group is a straight line, and
the at least two straight lines are arranged at a predetermined distance.

3. The endoscopic system according to claim 2, wherein the at least two straight lines are parallel to each other.

4. The endoscopic system according to claim 3, wherein the at least two straight lines are different in at least one of a color of the line, a density of shading of the line, a thickness of the line, and a line type.

5. The endoscopic system according to claim 1, wherein:
each of at least two of the plurality of balloon indices of the first index group is a convex portion, and
the at least two convex portions project linearly toward an interior portion of the balloon and are arranged at predetermined intervals.

6. The endoscopic system according to claim 5, wherein the at least two convex portions are parallel to each other.

7. The endoscopic system according to claim 6, wherein the at least two convex portions are different in at least one of a color of the convex portion, a density of shading of the color of the convex portion, a thickness of the convex portion, and a type of the convex portion.

8. The endoscopic system according to claim 1, further comprising a guide configured to guide an insertion direction of the sheath portion,
wherein:
the sheath portion includes a second index, and
the guide includes a third index configured to position with respect to the second index.

9. The endoscopic system according to claim 1, further comprising a guide configured to guide an insertion direction of the sheath portion,
wherein:
the sheath portion includes a first engagement,
the guide includes a second engagement which is configured to engage with the first engagement, and
the second engagement permits movement of the sheath portion in a longitudinal direction of the sheath portion, and restricts axial rotation of the sheath portion.

10. The endoscopic system according to claim 1, wherein:
the balloon is attached to the endoscope main body, and
the endoscope main body is, together with the balloon, removable from within the sheath portion in a direction opposite to the opening.

11. The endoscopic system according to claim 1, comprising a suction device that is configured to connect to the sheath portion from which the endoscope main body and the balloon have been removed, and is capable of suctioning an interior portion of a sinus via the sheath portion.

12. The endoscopic system according to claim 1, comprising an injection device that is configured to connect to the sheath portion from which the endoscope main body and the balloon have been removed, and is capable of injecting a liquid into the sinus via the sheath portion.

13. The endoscopic system according to claim 1, wherein the sinus is a paranasal sinus.

14. A sheath which allows an endoscope to be inserted therethrough, comprising:
a sheath portion including an opening; and
a balloon having translucency covering the opening and configured to inflate and deflate, the balloon including a first index group having a plurality of balloon indices which are adjacent to and different from each other.

15. The sheath according to claim 14, wherein an interior portion of the balloon is continuous with the opening of the sheath portion and an interior portion of the sheath portion.

16. The sheath according to claim 15, wherein an end portion of the balloon is attached to a distal end of the sheath portion including the opening.

17. A method of using an endoscopic system comprising:
a sheath portion including an opening;
a balloon including a first index group having a plurality of balloon indices; and
an endoscope main body configured to be retracted into the opening,
the method comprising:
inflating the balloon;
projecting the endoscope main body from the opening so as to allow the endoscope main body to be located inside the inflated balloon; and
observing an interior portion of a sinus with the endoscope main body with reference to the plurality of balloon indices of the first index group.

18. The method according to claim 17, comprising:
removing the endoscope main body, together with the balloon, from within the sheath portion in a direction opposite to the opening; and
performing a predetermined procedure on the interior portion of the sinus via the sheath portion from which the endoscope main body and the balloon have been removed.

19. The method according to claim 18, wherein
the performing the predetermined procedure includes suctioning content in the sinus via the sheath portion.

20. The method according to claim 18, wherein
the performing the predetermined procedure includes injecting a liquid into the sinus via the sheath portion.
